(19) United States
(12) Patent Application Publication
(10) Pub. No.: US 2007/0037987 A1
(43) Pub. Date: Feb. 15, 2007

(54) Preparation of 4,5-diamino-1-(substituted)-pyrazole and acid addition salts thereof

(76) Inventors: Kim Steven Chamberlin, Kingsport, TN (US); Charles Everette Kelly, Kingsport, TN (US)

Correspondence Address: Eastman Chemical Company, P.O. Box 511, Kingsport, TN, 37662-5075, US

(21) Appl. No.: 11/202,456

(22) Filed: Aug. 12, 2005

(51) Int. Cl. C07D 231/38

(52) U.S. Cl. 548/371.4

(57) ABSTRACT

Disclosed is an improved process for the preparation of 4,5-diamino-1-(substituted)pyrazoles and acid addition salts thereof by coupling the corresponding 5-amino-1-(substituted)pyrazole with an aromatic diazonium compound to produce an intermediate azo compound and contacting the intermediate azo compound with hydrogen in the presence of a hydrogenation catalyst to produce 4,5-diamino-1-(substituted)pyrazoles. Also disclosed is a decarboxylation process for the preparation of a 5-amino-1-(substituted)pyrazole by heating a solution comprising a 5-amino-4-carboxy-1-(substituted)pyrazole, an inorganic acid and an inert solvent.

### FIELD OF THE INVENTION

This invention pertains to an improved process for the preparation of 4,5-diamino-1-(substituted)pyrazoles and acid addition salts thereof such as the addition salt from sulfuric acid. More specifically, this invention pertains to the preparation of 4,5-diamino-1-(substituted)pyrazoles and acid addition salts thereof by coupling the corresponding 5-amino-1-(substituted)pyrazole with an aromatic diazonium compound to produce an intermediate azo compound and contacting the intermediate azo compound with hydrogen in the presence of a hydrogenation catalyst to produce 4,5-diamino-1-(substituted)pyrazoles. The present invention also pertains to the azo compounds produced as intermediates in the improved process. Another embodiment of the present invention pertains to the preparation of a 5-amino-1-(substituted)pyrazole by heating a solution comprising a 5-amino-4-carboxy-1-(substituted)pyrazole, an inorganic acid and a solvent selected from water, an alkanol or a mixture thereof.

### BACKGROUND OF THE INVENTION

1-Substituted-4,5-diaminopyrazoles and their addition salts are known to be especially useful as developers in combination with various couplers and an oxidizing agent for coloring keratinous fibers, particularly human hair. See, for example, U.S. Pat. Nos. 5,931,973; 5,769,902; 5,718,731; and 5,663,366. U.S. Pat. No. 5,663,366 describes the preparation of sulfuric acid salts of 1-substituted-4,5-diaminopyrazoles by a six-step process starting with pyrazole, a compound that is hydroscopic and an irritant, making it difficult to handle. In this process, the pyrazole is first nitrated to produce 4-nitropyrazole using a mixture of sulfuric and nitric acids. Subsequent bromination provides 3,5-dibromo-4-nitropyrazole. The 3,5-dibromo-4-nitropyrazole then is hydroxyethylated using 1-bromo-2-hydroxyethane with sodium hydride in N,N-dimethylformamide as a solvent to give 3,5-dibromo-1-(2-hydroxyethyl)-4-nitropyrazole which is then reacted with a large excess of benzylamine to provide 5-benzylamino-3-bromo-1-(2-hydroxyethyl)-4-nitropyrazole. This intermediate is recrystallized from toluene/ligroine (1:1). Finally, the product is obtained by a complicated hydrogenation using 10% palladium-on-carbon (Pd/C). Under the reduction conditions, the 3-bromo group is removed along with the benzyl group while the nitro group is reduced to an amine. The product then is isolated as the sulfuric or hydrochloric salt. It is known that halogen atoms such as bromine or chlorine deactivate noble metal catalysts, thus causing problems in reusing the metal catalysts. This overall procedure requires the isolation of at least five products, thus adding considerable handling costs and also requires a recrystallization and the handling of very toxic reagents such as nitric acid and bromine.

DE 3432983 describes the preparation of 4,5-diamino-1-substituted pyrazole hydrochloride (1): 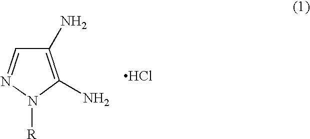
wherein R is benzyl; benzyl substituted with halogen or hydroxyl; C₁-C₈-alkyl; C₁-C₄-alkyl substituted with hydroxy. The hydrochloride salts are not isolated but are reacted to give 1,5-disubstituted-1H-pyrazolo-(3,4-b)-pyrazines. In the process of DE 3432983, ethyl (ethoxymethylene)-cyanoacetate is reacted with hydrazine hydrochloride in the presence of a sodium alkoxide in methanol to provide an ethyl ester of 5-amino-1-(substituted)-pyrazole-4-carboxylic acid. The methanol is removed by distillation and replaced with acetone to allow the removal of sodium chloride. The acetone is removed by distillation and replaced with ethanol to facilitate the isolation of the solid product. After isolation, the ester compound is placed in methanol and hydrolyzed to the acid by heating in the presence of 10% aqueous sodium hydroxide solution. Methanol is removed by distillation and the residue dissolved in dilute hydrochloric acid to give the solid 5-amino-4-carboxy-1-(substituted)-pyrazole which is recrystallized from methanol. Decarboxylation is accomplished by heating the 5-amino-4-carboxy-1-(substituted)-pyrazole at 160 to 170° C. and the resulting 5-amino-1-(substituted)pyrazole is distilled under vacuum and then nitrosated in ethanol containing hydrochloric acid by the addition of isoamyl nitrite. The solid 5-amino-4-nitroso-1-(substituted)-pyrazole is isolated and recrystallized, redissolved in methanol and hydrogenated in the presence of pallium-on-carbon catalyst. The catalyst is removed by filtration and the 4,5-diamino--1-(substituted)pyrazole hydrochloride is isolated by removing methanol or reacted directly in methanol without isolation to provide the 1,5-di(substituted)-1H-pyrazolo-(3,4-b)-pyrazines.

Example 4 of U.S. Pat. No. 2,989,537 discloses the preparation of solid 5-amino-4-carboxy-1-(2-hydroxyethyl)pyrazole by reacting ethyl (ethoxymethylene)cyanoacetate with 2-hydroxyethylhydrazine in ethanol and distilling the product under vacuum at high temperature. The isolated solid product then is hydrolyzed by heating with 2N aqueous sodium hydroxide followed by acidification with 6N hydrochloric acid to give 5-amino-4-carboxy-1-(2-hydroxyethyl)-pyrazole. This hydrolysis procedure leaves undissolved material which must be removed by filtration. The carboxy intermediate is heated to 160 to 170° C. to complete the decarboxylation and the 5-amino-1-(2-hydroxyethyl)-pyrazole is purified by distillation under vacuum at high temperature.

U.S. Pat. No. 6,452,019 discloses the preparation of 4,5-diamino-1-(2-hydroxyethyl)-pyrazole by the following process steps: 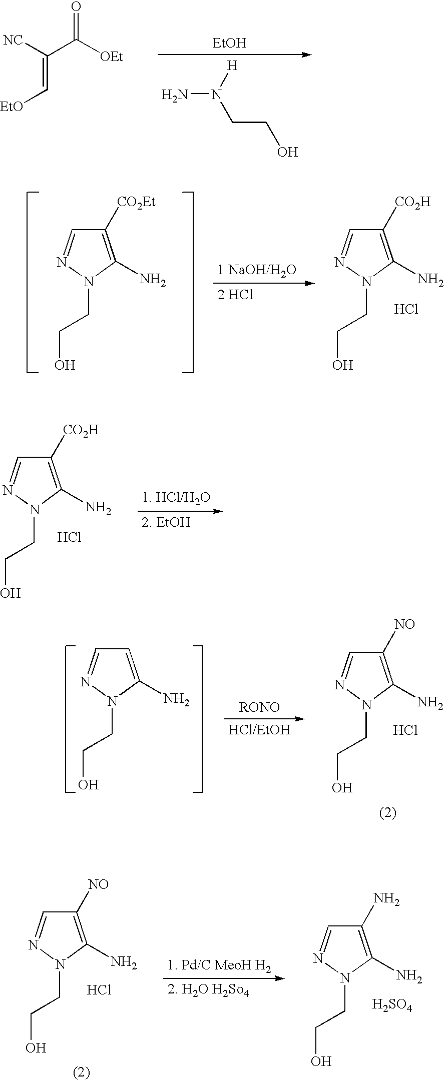
The advantage of this procedure is that it avoids multiple isolations, recrystallizations, high temperature vacuum distillations, etc. The disadvantage is in the nitrosation step due to the occurrence of diazotization of the amino group at the same rate as the nitrosation occurs. Following diazotization the diazonium salt couples with the starting material to give (3). This upsets the effective stoichiometry which leads (3) to diazotize and react with the chloride present in a Sandmeyer reaction to give (4). Higher concentrations of unidentified impurities also are present. 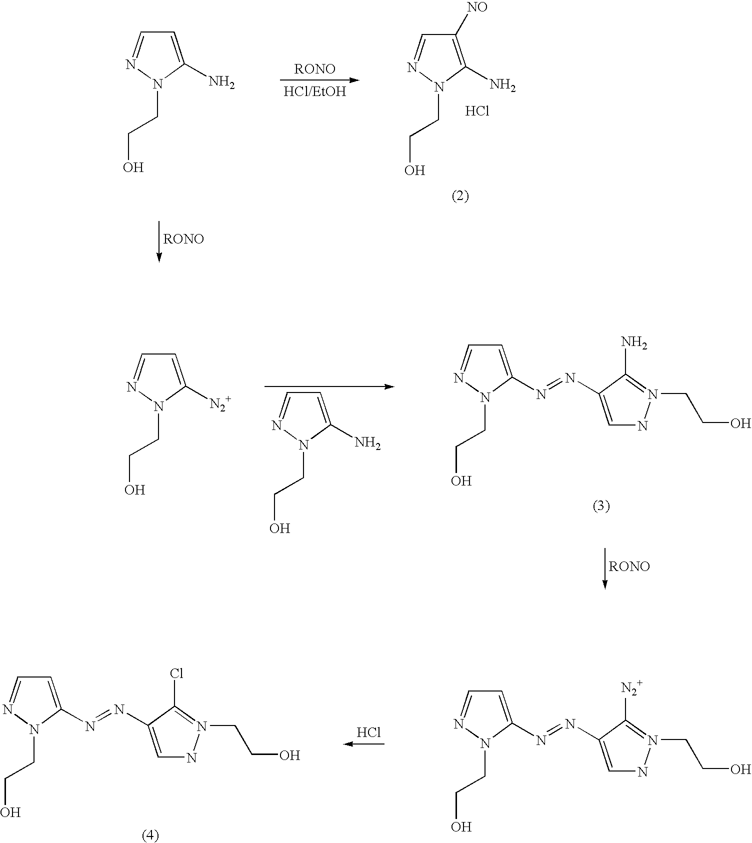
Upon hydrogenation a myriad of impurities are produced that suppress the crystallization of the desired product often with no product being isolated.

### BRIEF SUMMARY OF THE INVENTION

We have developed an improved process for the preparation of 4,5-diamino-1-(substituted)pyrazoles and acid addition salts thereof such as the addition salt from sulfuric acid. The present invention provides a process for the preparation of a compound having the formula 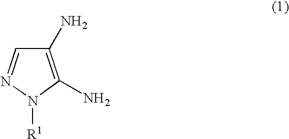
or an acid addition salt thereof by the steps comprising:
(1) contacting a compound having formula (2): 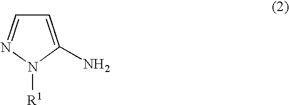
   or an acid addition salt thereof with diazonium salt (3) having the formula R²—NN⁺X⁻ in the presence of an inert solvent to produce azo compound (4) having the formula: 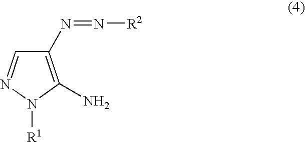
   or an acid addition salt thereof and
(2) contacting azo compound (4) with hydrogen in the presence of an insoluble hydrogenation catalyst under hydrogenation conditions of temperature and pressure to produce a compound (1);
   wherein
   R¹ is unsubstituted or substituted alkyl;
   R² is an aromatic, carbocyclic or heterocyclic residue of an aromatic diazotizable amine; and
   X is the anion residue of an acid.
      A second embodiment of the present invention comprises the azo compounds having formula (4) and acid addition salts thereof. A third embodiment of our invention comprises a decarboxylation process for the preparation of a 5-amino-1-(substituted)pyrazole or an acid addition salts thereof by heating a solution comprising (i) a 5-amino-4-carboxy-1-(substituted)pyrazole or an acid addition salt thereof, (ii) an inorganic acid and a solvent selected from water, an alkanol or a mixture thereof at a temperature of about 40 to 100° C.

The process provided by the present invention overcomes the problem of by-product formation resulting for the nitrosation step of the process utilized in the process of U.S. Pat. No. 6,452,019. Our novel process also is safer to operate since the nitroso intermediate of U.S. Pat. No. 6,452,019 exhibits an exotherm of nearly 900 Joules per gram starting at 81° C. by differential scanning calorimetry. In contrast, the analogous azo intermediate (4) of the present invention exhibits only a moderate exotherm beginning at 175° C.

### DETAILED DESCRIPTION

The first step of our novel process involves contacting a compound having formula (2): 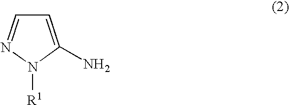
or an acid addition salt thereof with a diazonium salt having the formula R²—NN⁺X⁼ (3) in the presence of an inert solvent to produce an azo compound having the formula: 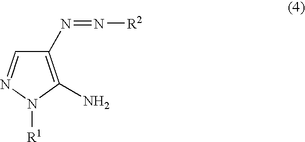
or an acid addition salt thereof. Pyrazole starting material (2) may be obtained from various known processes including the processes described in certain of the prior art discussed hereinabove. The procedures described in U.S. Pat. No. 6,452,019 are especially useful in combination with the processes provided by the present invention. Pyrazole starting material (2) preferably is prepared by the improved decarboxylation process described herein. The substituents represented by R¹ include unsubstituted and substituted alkyl, e.g., straight- and branched-chain alkyl containing from 1 to 8 carbon atoms and straight- and branched-chain alkyl containing from 1 to 8 carbon atoms substituted with hydroxyl, halogen or alkylphenyl. R¹ preferably represents a 2-hydroxyethyl group. Pyrazole starting material (2) preferably is a hydrohalide addition salt, especially a hydrochloride salt.

Pyrazole (2) is reacted (or coupled) with diazonium salt (3) in the presence of an inert solvent at a temperature of about −5 to 10° C., preferably about 0 to 5° C., to produce intermediate azo compound (4). The inert solvent preferably is water or an alkanol, e.g., an alkanol containing 1 to 3 carbon atoms. The relative amounts of pyrazole (2) and diazonium salt (3) employed in the first step typically will be in the range of about 1.2 to 1.0 moles diazonium salt (3) per mole of pyrazole (2). The use of an excess diazonium salt (3) causes formation of by-product (VI) which upon hydrogenation gives desired product (1) plus an aromatic hydrazine.

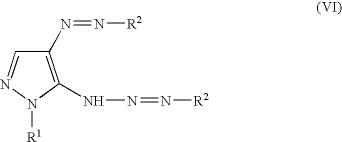

Diazonium salt (3) may be prepared according to known procedures by diazotizing a diazotizable, aromatic amine having the formula R²—NH₂ (5). Typical diazotization procedures involve contacting amine (5) with a nitrite in the presence of an acid and an inert solvent at a temperature of about −5 to 10° C., preferably about 0 to 5° C. Examples of nitrite compounds include alkali metal nitrites, e.g., sodium nitrite, nitrosyl sulfuric acid and alkyl nitrites, e.g. C₄ to C₈ alkyl nitrites, especially isoamyl nitrite. The diazotiation preferably is carried out in water or an alkanol containing 1 to 3 carbon atoms. R² is an aromatic, carbocyclic or heterocyclic residue of an aromatic diazotizable amine; Examples of the radicals represented by R² include unsubstituted and substituted phenyl, unsubstituted and substituted 1- and 2-naphthyl, unsubstituted and substituted 2-thiazolyl, unsubstituted and substituted 2-benzothiazolyl, unsubstituted and substituted 3-benxisothiazolyl, unsubstituted and substituted 2-1,3,4-thiadiazolyl, unsubstituted and substituted 5-1,2,4-thiadiazolyl, and similar carbocyclic and heterocyclic aromatic amines. Amine (5) preferably is aniline or sulfanilic acid (p-sulfoanile). Anion X⁻ of diazonium salt (3) most commonly is a halide, e.g., Cl, or a sulfate, e.g., SO₄.

Step (1) of the process of our invention conveniently is carried out by combining the solution of diazonium (3) in water or a C₁ to C₃ alkanol with a solution of an acid addition salt, e.g., a hydrochloric acid salt, of pyrazole intermediate (2) in water or a C₁ to C₃ alkanol while maintaining the temperature of the individual and combined solution at about −5 to 10° C., preferably about 0 to 5° C., to form azo compound (4). Azo compound (4) forms as a solid in the combined solution and normally is recovered by conventional liquid/solid separation techniques such as filtration, centrifugation or the like. If necessary, the azo compound thus recovered may be purified, for example, by washing with a C₁ to C₃ alkanol or by recrystallization be conventional procedures.

The second step of our novel process comprises contacting azo compound (4) with hydrogen in the presence of an inert solvent and an insoluble hydrogenation catalyst under hydrogenation conditions of temperature and pressure to produce 4,5-diamino-1-(substituted)pyrazole compound (1). The hydrogenation catalyst may be selected from a variety of known noble metal hydrogenation catalysts. Examples of such hydrogenation catalysts include Raney nickel and supported catalysts comprising a noble metal such as platinum, palladium, and the like deposited on a catalyst support material. Supported catalyst comprising about 0.1 to 10 weight percent platinum or palladium on carbon have been found to be an effective catalyst for the hydrogenation of step (2). In the hydrogenation azo compound (4) is dissolved in an inert solvent such as an alkanol containing 1 to 3 carbon atoms. The weight ratio of azo compound (4):inert solvent typically is about 0.15:1 to 0.45:1. The amount of hydrogenation catalyst typically present provides a catalyst:azo compound (4):weight ratio of about 0.03:1 to 0.3:1 depending upon the particular catalyst employed. The hydrogenation conditions of temperature and pressure comprise temperatures in the range of about 20 to 70° C. and hydrogen pressures in the range of about 3.5 to 40 bars gauge (barg—approximately 50 to 600 pounds per square inch—psig). The preferred operating conditions comprise temperatures in the range of about 55 to 70° C. and hydrogen pressures in the range of about 35 to 40 barg (approximately 500 to 600 psig). Upon completion of the hydrogenation, the hydrogenation product mixture is allowed to cool to ambient temperature and the catalyst is removed, e.g., by filtration. Sulfuric acid may be added to cause precipitation of the sulfuric acid addition salt of 4,5-diamino-1-(substituted)pyrazole which may be recovered by filtration. Alternatively, the 4,5-diamino-1-(substituted)pyrazole hydrochloride product may be reacted to give 1,5-disubstituted-1H-pyrazolo-(3,4-b)-pyrazines.

The present invention also provides a process for the preparation of pyrazole compound (2) having the formula: 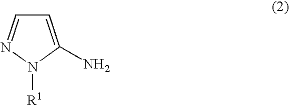
which comprises heating a solution comprising (i) a carboxy compound having the formula: 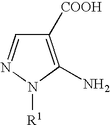
(ii) an inorganic acid and (iii) an inert solvent. The solvent preferably is a mixture of water and an alkanol containing 1 to 3 carbon atoms resulting from the mixing of an aqueous solution of an inorganic acid with an alkanol. The decarboxylation process may be carried out at a temperature of about 40 to 100° C. The concentration of the carboxypyrazole reactant in the inert solvent may be in the range of about 5 to 35 weight percent based on the total weight of the reaction mixture. The concentration of the inorganic acid in the solvent typically is in the range of about 1 to 10 weight percent, based on the total weight of the reaction mixture. The inorganic acid preferably is a hydrogen halide, e.g., hydrochloric acid, or sulfuric acid. The time required for complete or substantially complete decarboxylation of the carboxy compound to produce pyrazole compound (2) varied depending upon the temperature that may be achieved with the solvent or solvent mixture selected. The decarboxylation process preferably is carried out by heating a solution comprising (i) about 5 to 35 weight percent carboxypyrazole reactant, (ii) about i to 10 weight percent hydrogen chloride and (iii) a solvent comprising a mixture of water and an alkanol containing 2 or 3 carbon atoms at a temperature of about 40 to 100° C.

### EXAMPLE

The process provided by the present invention is further illustrated by the following example. All percentages given in the example are by weight unless otherwise specified.

5-Amino-4-Carboxy-1-(2′-Hydroxyethyl)-Pyrazole Hydrochloride

Ethanol (80 mL) and 2-hydroxyethylhydrazine (245.04 g, 90% assay) were combined in a stirred round-bottom flask, A solution of ethyl (ethoxymethylene)cyanoacetate (470.4 g) in ethanol (300 mL) was added allowing the reaction mixture to exotherm to 75-80° C. The mixture was held for three hours at 75-80° C. and then cooled to ambient temperature. 50% aqueous sodium hydroxide (282.2 g) was diluted with water (94 mL) and added to the reaction mixture. The resulting mixture was heated to 75-80° C. and held three hours. Water (300 mL) was added and the solution was cooled to 10-15° C. The hydrochloride addition salt of 5-amino-4-carboxy-1-(2′-hydroxyethyl)pyrazole was precipitated by the addition of aqueous hydrochloric acid (37%, 320 mL). The product was isolated by filtration, washed twice with 2-propanol (2×400 mL) and once with ethyl acetate (400 mL). The product was dried in a 40° C. vacuum oven. The yield was 324.2 g, 53.6% of the theoretical. Weight percent assay (purity) by pmr was 100%.

5-Amino-1-(2′-Hydroxyethyl)-4-Phenylazopyrazole Hydrochloride

5-Amino-4-carboxy-(2′-hydroxyethyl)pyrazole hydrochloride (62.08 g, 0.298 moles 100% basis) was added to ethanol (116 mL). Hydrochloric acid (37%, 23.2 mL) was added and the mixture was refluxed (80° C.) for one hour. An analytical sample showed that decarboxylation was complete. The mixture was cooled to 0 to 5° C. Hydrochloric acid (37% 25.0 mL) was added to a mixture of aniline (27.72 g, 0.298 mole) in ethanol (200 mL). The mixture was cooled to 0-5° C. and isoamyl nitrite (40 mL, 0.298 mole) was added while maintaining the temperature at 0-5° C. The resulting diazonium salt was held one hour at 0-5° C. and then was slowly added to the coupler at 0-5° C. The mixture was stirred overnight while allowing it to come to ambient temperature. The product was collected by filtration and washed with isopropyl alcohol. The product was vacuum dried at 60° C. The yield was 59.36 g 74.5% of the theoretical. Area percent assay by hplc was 98.6%.

4,5-Diamino-1-(2′Hydroxyethyl)pyrazole Sulfuric Acid Salt

5-Amino-4-phenylazo-(2′-hydroxyethyl)-pyrazole hydrochloride (59.3 g 100% basis, 0.221 mole) was slurried in methanol (150 mL). Platinum on carbon (1.0%, 1.0 g) was added. And the mixture was transferred to an autoclave. The reduction was done under hydrogen at 60° C. and 14.35 bar gauge (600 pounds per square inch—psig). The mixture was cooled to ambient temperature and clarified. Sulfuric acid (98%, 24.58 g) was added at <30° C. The product was isolated by filtration and washed with methanol (3×20 mL) and dried. The yield was 27.54 g (51.7% of the theoretical based on 0.221 mole of phenylazo intermediate (4)). Assay was 99.30 wt % by hplc.

The invention has been described in detail with particular reference to preferred embodiments thereof, but it will be understood that variations and modifications can be effected within the spirit and scope of the invention.

We claim:

1. Process for the preparation of a compound having the formula 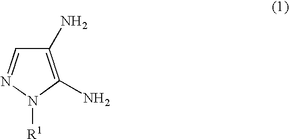
and acid addition salts thereof by the steps comprising:
(1) contacting a compound having formula (2): 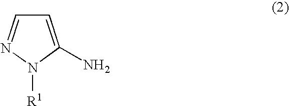
or an acid addition salt thereof with diazonium salt (3) having the formula R²—NN⁺X⁻ in the presence of an inert solvent to produce azo compound (4) having the formula: 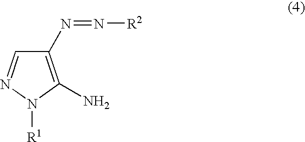
and
(2) contacting azo compound (4) with hydrogen in the presence of an insoluble hydrogenation catalyst under hydrogenation conditions of temperature and pressure to produce a compound (1);
wherein
R¹ is unsubstituted or substituted alkyl;
R² is an aromatic, carbocyclic or heterocyclic residue of an aromatic diazotizable amine; and
X is the anion residue of an acid.

2. Process according to claim 1 wherein step (1) is carried out at a temperature of −5 to 10° C. in the presence of an inert solvent selected from water and an alkanol.

3. Process according to claim 1 wherein step (1) is carried out at a temperature of 0 to 50° C. in the presence of an inert solvent selected from water and an alkanol containing 1 to 3 carbon atoms wherein the amounts of pyrazole (2) and diazonium salt (3) employed are in the range of about 1.2 to 1.0 moles diazonium salt (3) per mole pyrazole (2).

4. Process according to claim 1 wherein step (1) is carried out at a temperature of −5 to 10° C. in the presence of an inert solvent selected from water and an alkanol and step (2) comprises contacting azo compound (4) with hydrogen in the presence of an insoluble hydrogenation catalyst selected from Raney nickel and a nobel metal deposited on a catalyst support material at a temperature of about 20 to 70° C. and a hydrogen pressure of about 3.5 to 40 bars gauge.

5. Process according to claim 1 wherein step (1) is carried out at a temperature of 0 to 5° C. in the presence of an inert solvent selected from water and an alkanol containing 1 to 3 carbon atoms wherein the amounts of pyrazole (2) and diazonium salt (3) employed are in the range of about 1.2 to 1.0 moles diazonium salt (3) per mole pyrazole (2) and step (2) comprises contacting azo compound (4) with hydrogen in the presence of an insoluble hydrogenation catalyst selected from supported catalysts comprising 0.1 to 10 weight percent platinum of palladium on carbon at a temperature of about 55 to 70° C. and a hydrogen pressure of about 35 to 40 bars gauge.

6. Process for the preparation of a hydrochloride salt of a compound having the formula 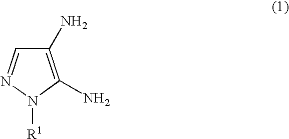
by the steps comprising:
(1) contacting a hydrochloride addition salt of a compound having formula (2): 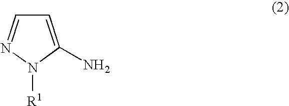
with diazonium salt (3) having the formula R²—NN⁺X⁻ at a temperature of −5 to 10° C. in the presence of an inert solvent selected from water and an alkanol to produce a hydrochloride salt of an azo compound (4) having the formula: 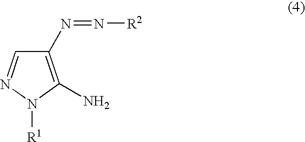
and
(2) contacting azo compound (4) with hydrogen in the presence of an insoluble hydrogenation catalyst selected from Raney nickel and a nobel metal deposited on a catalyst support material at a temperature of about 20 to 70° C. and a hydrogen pressure of about 3.5 to 40 bars gauge;
wherein
R¹ is 2-hydroxyethyl;
R² is phenyl or substituted phenyl; and
X is halide or sulfate anion.

7. Process according to claim 6 wherein step (1) is carried out at a temperature of 0 to 5° C. in the presence of an inert solvent selected from water and an alkanol containing 1 to 3 carbon atoms wherein the amounts of pyrazole (2) and diazonium salt (3) employed are in the range of about 1.2 to 1.0 moles diazonium salt (3) per mole pyrazole (2); step (2) comprises contacting azo compound (4) with hydrogen in the presence of an insoluble hydrogenation catalyst selected from supported catalysts comprising 0.1 to 10 weight percent platinum of palladium on carbon at a temperature of about 55 to 70° C. and a hydrogen pressure of about 35 to 40 bars gauge; and R² is phenyl or sulfophenyl.

8. An azo compound having the formula: 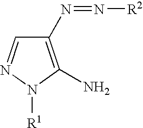
and acid addition salts thereof wherein
R¹ is unsubstituted or substituted alkyl; and
R² is an aromatic, carbocyclic or heterocyclic residue of an aromatic diazotizable amine.

9. A hydrochloride salt of an azo compound defined in claim 8 wherein R¹ is 3-hydroxyethyl and R² is phenyl or substituted phenyl.

10. Process for the preparation of an acid addition salt of a pyrazole compound (2) having the formula: 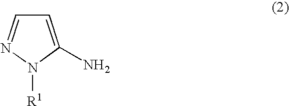
which comprises heating a solution comprising (i) a carboxy reactant having the formula: 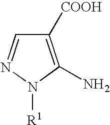
(ii) an inorganic acid and (iii) an inert solvent at a temperature of about 40 to 100° C.; wherein R¹ is unsubstituted or substituted alkyl.

11. A process according to claim 10 wherein the inert solvent is a mixture of water and an alkanol containing 1 to 3 carbon atoms and the concentration of the inorganic acid is about 1 to 10 weight percent based on the total weight of the reaction mixture.

12. A process according to claim 10 for the preparation of the hydrochloride of a pyrazole compound (2) having the formula: 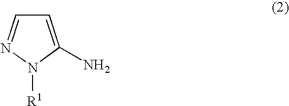
which comprises heating a solution comprising (i) about 5 to 35 weight percent of a carboxy reactant having the formula: 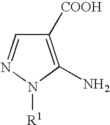
(ii)about 1 to 10 weight percent hydrogen chloride and (iii) an inert solvent comprising a mixture of water and an alkanol containing 1 to 3 carbon atoms at a temperature of about 40 to 100° C.; wherein R¹ is 2-hydoxyethyl.
